SEQUENCE LISTING (19 sequences)

SEQ ID NO: 1 - DNA, 24 bp, Artificial Sequence
  Synthetic Primer
aacggatcccgccgcttctttgag

SEQ ID NO: 2 - DNA, 30 bp, Artificial Sequence
  Synthetic Primer
aacggatccaaaaaaaatccttagctttcg

SEQ ID NO: 3 - DNA, 53 bp, Artificial Sequence
  Synthetic Primer
aagatctcatcaccatcaccatcactaagcttaattagctgagcttggactcc

SEQ ID NO: 4 - DNA, 35 bp, Artificial Sequence
  Synthetic Primer
gctcgagcatgcttggattctcaccaataaaaaac

SEQ ID NO: 5 - DNA, 37 bp, Artificial Sequence
  Synthetic Primer
ctctaaatccgcatggcaggggttcaaatccggcccg

SEQ ID NO: 6 - DNA, 37 bp, Artificial Sequence
  Synthetic Primer
cgggccggatttgaacccctgccatgcggatttagag

SEQ ID NO: 7 - DNA, 32 bp, Artificial Sequence
  Synthetic Primer
ggcaggggttcaaatcccctccgccggaccac

SEQ ID NO: 8 - DNA, 32 bp, Artificial Sequence
  Synthetic Primer
gtggtccggcggaggggatttgaacccctgcc

SEQ ID NO: 9 - DNA, 32 bp, Artificial Sequence
  Synthetic Primer
attccatatgggccaggccaaccacccaacag

SEQ ID NO: 10 - DNA, 54 bp, Artificial Sequence
  Synthetic Primer
ccacgtggcaccaatttccaaactgcatcaatgaatggtgtcacacagatgaac

SEQ ID NO: 11 - DNA, 32 bp, Artificial Sequence
  Synthetic Primer
cctaagctttgatccacgtggcaccaatttcc

SEQ ID NO: 12 - DNA, 61 bp, Artificial Sequence
  Synthetic Primer
gtataatagattcataaatttgattaaagaggagaaattaactatgggccaggccaaccac

SEQ ID NO: 13 - DNA, 35 bp, Artificial Sequence
  Synthetic Primer
ccacgtagatcttgatccacgtggcaccaatttcc

SEQ ID NO: 14 - DNA, 50 bp, Artificial Sequence
  Synthetic Primer
cagctcgggcccttgctttcaggaaaatttttcagtataatagattcata

SEQ ID NO: 15 - DNA, 29 bp, Artificial Sequence
  Synthetic Primer
gtagtgctgccccgttcctgaagaccaag

SEQ ID NO: 16 - DNA, 29 bp, Artificial Sequence
  Synthetic Primer
cttggtcttcaggaacggggcagcactac

SEQ ID NO: 17 - DNA, 32 bp, Artificial Sequence
  Synthetic Primer
gcaccttcctactgcagttcagcgagagcagc

SEQ ID NO: 18 - DNA, 32 bp, Artificial Sequence
  Synthetic Primer
gctgctctcgctgaactgcagtaggaaggtgc

SEQ ID NO: 19 - DNA, 29 bp, Artificial Sequence
  Synthetic Probe
agcttcatttcccgtaaatccctaaagct

(19) United States
(12) Patent Application Publication
(10) Pub. No.: US 2013/0078660 A1
(43) Pub. Date: Mar. 28, 2013

(54) METHODS AND COMPOSITIONS FOR DETECTING PROTEIN MODIFICATIONS

(71) Applicants: Lei Wang, San Diego, CA (US); Vanessa K. Lacey, San Diego, CA (US); Angela R. Parrish, New yok, NY (US)

(72) Inventors: Lei Wang, San Diego, CA (US); Vanessa K. Lacey, San Diego, CA (US); Angela R. Parrish, New yok, NY (US)

(73) Assignee: SALK INSTITUTE FOR BIOLOGICAL STUDIES, LA JOLLA, CA (US)

(21) Appl. No.: 13/636,558

(22) PCT Filed: Mar. 23, 2011
(86) PCT No.: PCT/US11/29680

§ 371 Date: Dec. 4, 2012

Related U.S. Application Data
(60) Provisional application No. 61/316,761, filed on Mar. 23, 2010.

(51) Int. Cl. C12Q 1/48

(52) U.S. Cl. CPC C12Q 1/485
USPC 435/15; 435/4

(57) ABSTRACT

Methods and compositions for detecting a protein modification in vitro and in vivo are disclosed. In certain embodiments, the protein modification detected is phosphorylation.

### RELATED APPLICATION

This application claims the benefit of priority of U.S. Provisional Application Ser. No. 61/316,761, filed Mar. 23, 2010. The foregoing application is incorporated herein by reference in its entirety.

### STATEMENT AS TO RIGHTS TO INVENTIONS MADE UNDER FEDERALLY SPONSORED RESEARCH OR DEVELOPMENT

The present invention was supported by a grant awarded by the US National Institutes of Health (1DP20D004744). The Government has certain rights in the invention.

### BACKGROUND OF THE INVENTION

Phosphorylation of Tyr, Ser or Thr by protein kinases regulates many intracellular signal transduction cascades, the aberration of which is involved in a variety of diseases such as cancer, autoimmune disorders, and cardiovascular diseases¹. The ability to monitor the phosphorylation events would provide valuable information for understanding the regulation mechanisms and for developing effective therapeutics². Kinase activities can be optically reported using fluorescent sensors based on protein or peptide constructs. A substrate peptide or domain is used for sensing the kinase activity, phosphorylation of which leads to fluorescence change in the genetically attached fluorescent proteins or chemically introduced small molecule fluorophores. The main advantage of peptide-base reporters is their large fluorescence change, which has proven extremely sensitive in in vitro assays³ yet introducing the reporter into cells is challenging. Protein-based reporters, though with smaller responses, are genetically encoded and have revealed novel spatiotemporal information regarding a variety of signaling in living cells.

There are over 500 putative kinases in the human genome, and closely related kinases tend to catalyze the phosphorylation of the same peptide⁵. In addition to the sequences proximal to the phosphorylation residue, many kinases also derive specificity on distal residues of the substrate protein involved in docking or other interactions⁶. When only a short substrate peptide is used for recognition, as in most of the current fluorescent reporters, specificity for the target kinase becomes a great challenge⁷. On the other hand, a kinase usually has multiple substrates. The detection of the kinase activity shed no or limited information on the phosphorylation state of a specific substrate protein. Moreover, subcellular location, trafficking and lifetime of a substrate protein are difficult to be faithfully replicated by the comparatively simplified peptide or domain sensor elements. The present invention includes a fluorescent reporter of the phosphorylation status of a target protein, so as to enable the optical investigation of protein phosphorylation on the level of substrate in addition to of kinase.

### SUMMARY OF THE INVENTION

In certain embodiments, the invention relates to a method for the detection of a modification to a target protein, comprising (a) contacting a target protein comprising an unnatural amino acid with a modifying enzyme; and (b) assaying for a detectable signal from the unnatural amino acid in (a) after the target protein of (a) has been contacted with the modifying enzyme, wherein if the modifying enzyme modifies the target protein, the unnatural amino acid generates a detectable signal, thereby indicating that the target protein has been modified.

In some embodiments, the detectable signal is fluorescence. In some embodiments, the unnatural amino acid is 7HC.

In some embodiments, the target protein comprises an SH2 domain. In certain embodiments, the target protein is STAT3.

In certain embodiments, the modification to the target protein is phosphorylation. In some embodiments, the modifying enzyme is a kinase.

In some embodiments, the target protein comprising the unnatural amino acid is expressed in a host cell selected from the group consisting of bacterial, insect, and mammalian cells. In certain embodiments, the host cell is a bacterial cell, such as an E. coli cell. In other embodiments, the host cell is a mammalian cell, such as a HepG2 cell. In yet other embodiments, the method further comprises contacting the host cell expressing the target protein with a physiological activator of the target protein. In a particular embodiment, the target protein is STAT3 and the physiological activator is IL-6.

In some embodiments, the unnatural amino acid generates a detectable signal as a result of a conformational change. In other embodiments, the unnatural amino acid generates a detectable signal as a result of a pH change.

### BRIEF DESCRIPTION OF THE DRAWINGS

FIG. 1. Reporting the phosphorylation status of a substrate protein using genetically encoded unnatural amino acids. (a) Schematic illustration of the strategy. The broken line indicates that the phosphorylation residue and the fluorescent unnatural amino acid can be from the same protein or separate proteins. (b) Crystal structure of the STAT3β homodimer. The two monomers are colored in pink and cyan and the DNA in gray (PDB 1BG1). (c) Zoomed in on the region framed in (b) to illustrate the location of pTyr705 (red) in relation to Trp564 (green). (d) Structure of L-(7-hydroxycoumarin-4-yl) ethylglycine (7HC), an unnatural amino acid with fluorescence sensitive to its environment.

FIG. 2. 7HC-STAT3β is phosphorylated and binds the consensus DNA sequence similar to wt STAT3β. (a) Western blot analysis of cell lysates from E. coli cells expressing pAIO-7HC and pRARE-2. Full length STAT3β was produced in the presence of 7HC. (b) Photograph of SDS-PAGE (8%) analysis of wt STAT3β, STAT3β (564TAG) expressed in the presence and absence of 7HC. The gel was exposed to 365 nm UV light. (c) Western blot of protein samples incubated with and without the Src kinase. 7HC-STAT3β as well as wt STAT3β were phosphorylated at Tyr705 by Src kinase. (d) EMSA using P-32 labeled hSIE DNA probe. Both wt and 7HC-STAT3β were upshifted indicating they both bound the hSIE probe.

FIG. 3. 7HC reports the phosphorylation status of STAT3β with reversibility and high sensitivity. (a) Fluorescence emission of 7HC-STAT3β before and after phosphorylation by Src kinase followed by dephosphorylation by CIP in HEPES buffer (pH=7.5). (b) Fluorescence emission of 7HC-STAT3β mutants before and after phosphorylation by Src kinase in HEPES buffer (pH=7.5). (c) Western blots for 7HC-STAT3β and mutants using an antibody against phosphorylated STAT3. Same amounts of cell lysate were loaded in each lane.

FIG. 4. 7HC in the 7HC-STAT3β protein experiences pH change upon phosphorylation. (a) Fluorescence excitation spectra of 7HC in aqueous buffer with emission recorded at 450 nm. (b) Fluorescence emission spectra of 7HC in aqueous buffer with excitation at 363 nm. (c) Fluorescence excitation spectra of 7HC-STAT3β with emission recorded at 450 nm. Broken and solid lines indicate 7HC-STAT3β protein before and after phosphorylation by Src kinase, respectively.

FIG. 5. 7HC-STAT3β reports the phosphorylation status of endogenous STAT3 from HepG2 cells and indicates a difference between cytoplasmic and nuclear STAT3. (a) Western blot showing that STAT3 was phosphorylated in the IL-6 activated HepG2 cells in both the nucleus and the cytoplasm. (b) Fluorescence increase of 7HC-STAT3β upon incubation with different cell lysates. The values (±s.e.m.) were: nuclear IL-6 (−) 1.4±0.2, nuclear IL-6 (+) 5.9±0.8, cytoplasmic IL-6 (−) 1.7±0.4, cytoplasmic IL-6 (+) 1.7±0.2. For all samples, n=3 from 3 independent batches of HepG2 cells. The IL-6 activated nuclear fraction was statistically different from the other 3 samples, whereas the other 3 samples were not statistically different from each other (Student's t-test, two-tailed, unpaired). (c) Fluorescence emission spectra of 7HC-STAT3β after incubation with the nuclear (left panel) and cytoplasmic (right panel) cell lysates of HepG2 cells. Only the nuclear fraction from IL-6 activated cells showed the characteristic double emission peak. Note there was a strong Raman peak at 402 nm in both cytoplasmic fractions due to their high protein concentration. (d) Fluorescence excitation spectra of 7HC-STAT3β after incubation with the nuclear (left panel) and cytoplasmic (right panel) cell lysates of HepG2 cells. Only the nuclear fraction from IL-6 activated cells showed excitation peak shift. (e) Western blot showing that 7HC-STAT3β was not phosphorylated by cell lysates. 7HC-STAT3β had the N-terminal domain deleted and thus ran at a different position from the endogenous STAT3. The blot was also probed with the penta-His antibody to detect the His6 tag appended at the C-terminus of 7HC-STAT3β. (f) Schematic illustration showing the difference between phosphorylated STAT3 localized in the cytoplasm vs. nucleus. Phosphorylated STAT3 in the cytoplasm is associated with various other proteins and thus cannot be exchanged with 7HC-STAT3β, whereas the nuclear STAT3 is more accessible for subunit exchange with 7HC-STAT3β to yield fluorescence increase.

### DETAILED DESCRIPTION OF THE INVENTION

### Definitions

The following definitions are provided to facilitate understanding of certain terms used herein and are not meant to limit the scope of the present disclosure.

“Nucleic acid” refers to deoxyribonucleotides or ribonucleotides and polymers thereof in either single- or double-stranded form, and complements thereof.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

The terms “identical” or percent “identity,” in the context of two or more nucleic acids, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The phrase “stringent hybridization conditions” refers to conditions under which a probe will hybridize to its target sequence, typically in a complex mixture of nucleic acids, but to not other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

A variety of methods of specific DNA and RNA measurement that use nucleic acid hybridization techniques are known to those of skill in the art (see, Sambrook, supra). Some methods involve electrophoretic separation (e.g., Southern blot for detecting DNA, and Northern blot for detecting RNA), but measurement of DNA and RNA can also be carried out in the absence of electrophoretic separation (e.g., by dot blot).

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBA, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation. It is understood that various detection probes, including Taqman and molecular beacon probes can be used to monitor amplification reaction products, e.g., in real time.

The word “polynucleotide” refers to a linear sequence of nucleotides. The nucleotides can be ribonucleotides, deoxyribonucleotides, or a mixture of both. Examples of polynucleotides contemplated herein include single and double stranded DNA, single and double stranded RNA (including miRNA), and hybrid molecules having mixtures of single and double stranded DNA and RNA.

The words “protein”, “peptide”, and “polypeptide” are used interchangeably to denote an amino acid polymer or a set of two or more interacting or bound amino acid polymers.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88).

The term “plasmid” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

Protein phosphorylation regulates numerous signaling cascades. Although kinase activity can be monitored with peptide- or fluorescent protein-based reporters, no fluorescence reporter exists for the phosphorylation status of a substrate protein. We developed a highly sensitive biosensor to reversibly report the phosphorylation of STAT3 by genetically incorporating 7-hydroxycoumarin into the phosphotyrosine binding pocket. The reporter showed a large fluorescence increase with characteristic emission and excitation in response to STAT3 phosphorylation by Src kinase and to STAT3 endogenously phosphorylated via the JAK-STAT pathway in cell lysates. Application of this reporter to human hepatoma HepG2 cells revealed a difference between phosphorylated STAT3 localized in the cytoplasm versus the nucleus. In certain embodiments, this approach is generally applicable to different STAT proteins and various SH2 domain-containing proteins to illuminate their functional roles in cell signaling.

Here we present a method to fluorescently report the phosphorylation status of a specific substrate protein. A small molecule fluorophore was genetically encoded in the format of an unnatural amino acid into the full length target protein to sense the phosphorylation change. We applied this method to monitor phosphorylation of the signal transducer and activator of transcription 3 (STAT3). A large fluorescence change was observed when the STAT3 probe was phosphorylated by Src kinase in vitro, and when it was bound to endogenously activated STAT3 in mammalian cell lysates. Our reporter further revealed a difference between phosphorylated STAT3 localized in the cytoplasm vs. nucleus. This strategy enables optical investigation of protein phosphorylation on the level of substrate with high specificity.

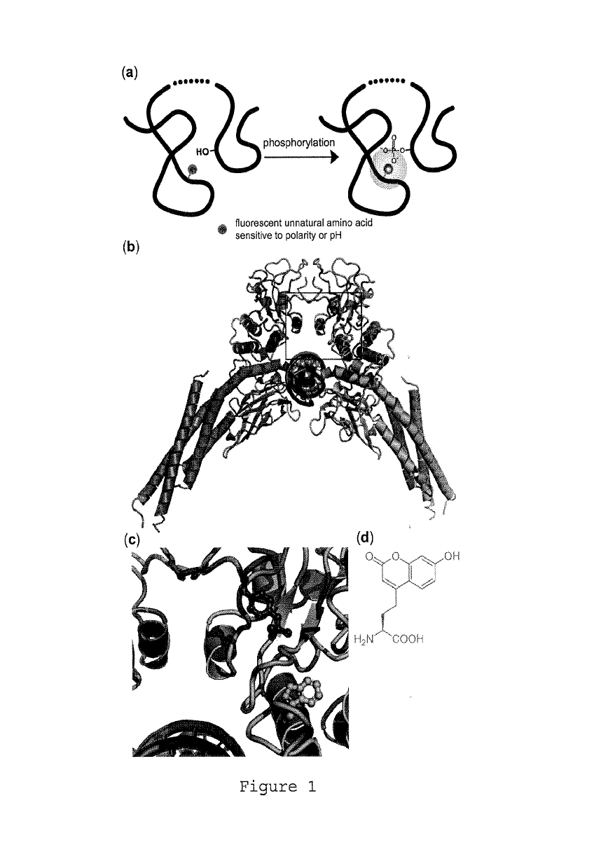

In certain embodiments, the instant invention provides a method to genetically incorporate a fluorescent unnatural amino acid into the target protein at a site close to the residue subject to phosphorylation (FIG. 1a). In some embodiments, upon phosphorylation, the introduction of the negatively charged phosphate group may change the local polarity or pH. In these embodiments, the fluorophore of the unnatural amino acid is typically designed to be sensitive to polarity or pH so that its fluorescence intensity and/or emission wavelength change in response to phosphorylation. By using a full-length substrate protein, one can incorporate the fluorescent unnatural amino acid at any site that is close to the phosphorylated residue in the tertiary structure, providing more flexibility in choosing the optimal sensor location than peptide-based methods. The closely positioned phosphorylated residue and the fluorescent amino acid can be within the same target protein, or in different proteins if the target protein is oligomeric or part of a protein complex.

The invention will now be further described by way of the following non-limiting examples.

### EXAMPLE 1

The signal transducer and activator of transcription 3 (STAT3) was employed as a target protein. STAT3 signaling plays a leading role in many oncogenic and developmental pathways, but the spatiotemporal and mechanistic details of STAT3 signaling are unclear (24). Upon phosphorylation on Tyr705, STAT3 dimerizes through the reciprocal binding of phosphotyrosine (pTyr) into the SH2 domain of an opposing monomer (FIG. 1b). The activated dimer translocates into the nucleus and binds consensus DNA sequences to regulate the expression of genes involved in oncogenesis, cell growth and differentiation. Without being bound to theory, we reasoned that binding of the negatively charged pTyr705 to the SH2 domain would change the microenvironment inside the binding pocket, most likely altering the pH due to the phosphate group. A pH-sensitive fluorophore should be able to detect this change optically. An example of a suitable fluorophore is 7-hydroxycoumarin, whose fluorescence intensity and excitation wavelength are pH-dependent with a pKa of ˜7.8 (25). Based on the crystal structure of STAT3β binding to DNA (FIG. 1b) (26), we selected Trp564 for mutation to L-(7-hydroxycoumarin-4-yl)ethylglycine (7HC, FIG. 1d). Trp564 is located at the 2nd layer of the SH2 binding pocket close to the pTyr of the opposing monomer, but distant from Tyr705 of the same monomer and outside of the DNA binding domain (FIG. 1c). Tip is also similar in size to 7HC. Collectively, these properties would minimize the potential interference from introducing 7HC.

### EXAMPLE 2

Genetic Incorporation of 7HC into STAT3β

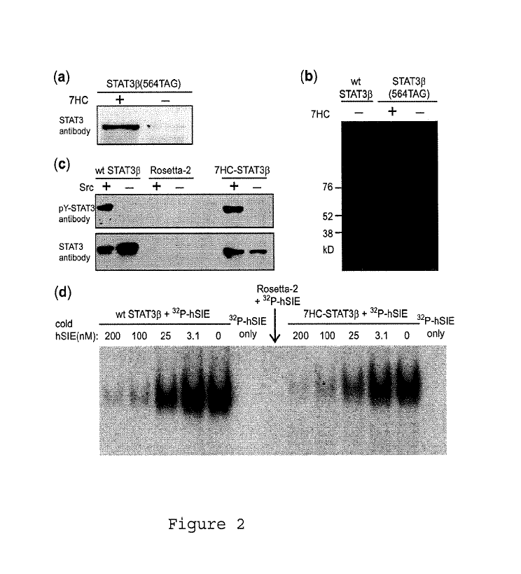

7HC was genetically incorporated into STAT3β in E. coli using an orthogonal tRNA/aminoacyl-tRNA synthetase pair reported by the Schultz group (27) to suppresses the amber TAG codon introduced at site 564. We started with the optimized pEVOL system (28) to express the orthogonal tRNA/synthetase combined with the pBAD vector to express the STAT3β gene, but no detectable 7HC-containing STAT3β was produced in E. coli, presumably due to multiple rare E. coli codons in the STAT3β gene. To solve this problem that can generally occur to the incorporation of unnatural amino acids into eukaryotic proteins expressed in E. coli, we constructed an all-in-one expression system (pAIO-7HC), which contains gene cassettes for the 7HC-specific synthetase, the suppressor tRNA_{CUA}^{Opt} , and the STAT3β (564TAG) gene. pAIO-7HC is compatible with the pRARE-2 plasmid, which expresses 7 rare tRNAs for enhanced expression of genes containing codons rarely used in E. coli. pAIO-7HC was transformed into Rosetta-2 (DE3) E. coli cells harboring pRARE-2. To verify the incorporation of 7HC, cell lysates were analyzed by Western blot using an antibody against STAT3 (FIG. 2a). Full-length STAT3β was observed only in the presence of 7HC. 7HC-containing STAT3β proteins (7HC-STAT3β) were purified with Ni-NTA chromatography with a yield of 56 μg/L (56±14 μg/L, n=4) of E. coli culture. In comparison, wild type STAT3β was purified with a yield of 750 μg/L of E. coli culture. A bright blue fluorescent band was observed for the purified 7HC-STAT3β on SDS-PAGE, but no fluorescent protein bands were observed for wt STAT3β or STAT3β (564TAG) expressed in the absence of 7HC (FIG. 2b).

To determine if Trp564 to 7HC substitution affects STAT3 function, purified 7HC-STAT3β protein was tested in vitro for its ability to be phosphorylated by the nonreceptor tyrosine kinase Src and its ability to bind the high-affinity sis-inducible element (hSIE) consensus DNA sequence in an electrophoretic mobility shift assay (EMSA). A 30 μL kinase reaction was carried out at 30° C. and stopped after 30 mins with EDTA. Ten μL was loaded onto a polyacrylamide gel for Western blot analysis and the remaining 20 μL was incubated with the P-32 labeled hSIE probe for the EMSA. The Western blot was first probed with an anti-phosphotyrosine STAT3 antibody (FIG. 2c). A clear band at the same molecular weight was seen both for phosphorylated wt STAT3β and for phosphorylated 7HC-STAT3β, with no signal in the control from Rosetta-2 extract or non-phosphorylated samples. The blot was stripped and re-probed with a STAT3 specific antibody, ensuring that comparable amounts of STAT3 were loaded for samples incubated with and without Src kinase. In the EMSA, a clear band shift at the same position was observed for both phosphorylated wt STAT3β and 7HC-STAT3β, but not for Rosetta-2 extract or the probe alone (FIG. 2d). These results indicate that 7HC-STAT3β, similar to wt STAT3β, can be phoshorylated and bind a consensus DNA sequence, suggesting that the replacement of Trp564 with 7HC does not significantly change STAT3 function. This is consistent with the location of residue 564, which is outside of the DNA-binding domain and away from the phosphorylation site Tyr705 in the same monomer.

### EXAMPLE 3

### Reporting Phosphorylation by Src Kinase

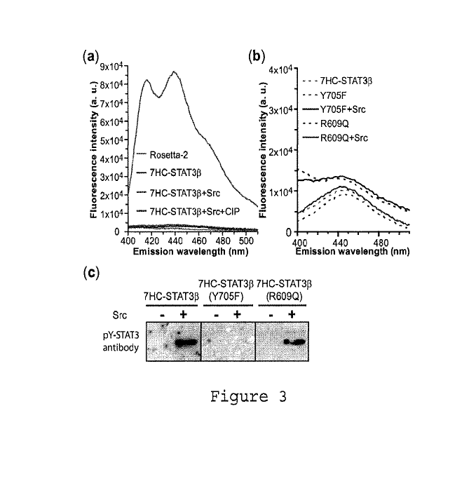

We next tested if the 7HC could sense and report the phosphorylation of STAT3β using fluorometry (FIG. 3a). Before phosphorylation, 7HC-STAT3β showed very weak fluorescence with a single emission peak at 448 nm. After incubation with Src kinase, the fluorescence intensity of 7HC-STAT3β increased markedly. A 13 (13±4.3, n=6) fold increase was detected for 20 nM of 7HC-STAT3β, indicating that the reporter is highly sensitive. In addition, a second emission peak emerged simultaneously at 416 nm. When calf intestinal phosphatase (CIP) was added into the phosphorylated 7HC-STAT3β sample, the fluorescence intensity dropped back to the level similar to unphosphorylated 7HC-STAT3β, indicating that the fluorescence change is reversible and dependent on phosphorylation status.

To confirm that the observed fluorescence change in 7HC-STAT3β was due to the phosphorylation of Tyr705, we made a 7HC-STAT3β (Y705F) mutant. The mutation of Tyr705 to Phe abolishes STAT3 phosphorylation by Src (FIG. 3c) (29). This mutant had the same fluorescence emission spectrum as the 7HC-STAT3β, and showed no fluorescence change upon incubation with Src (FIG. 3b). In addition, we made another mutant, 7HC-STAT3β (R609Q), which prevents binding of pTyr705 into the SH2 domain (30). This mutant could still be phosphorylated by Src kinase (FIG. 3c), yet exhibited no fluorescence change upon phosphorylation (FIG. 3b). These results indicate that the fluorescence change observed in 7HC-STAT3β can be attributed to the phosphorylation of Tyr705 and the subsequent binding of pTyr705 to the SH2 domain.

### EXAMPLE 4

### Phosphorylation Sensing Mechanism of the Reporter

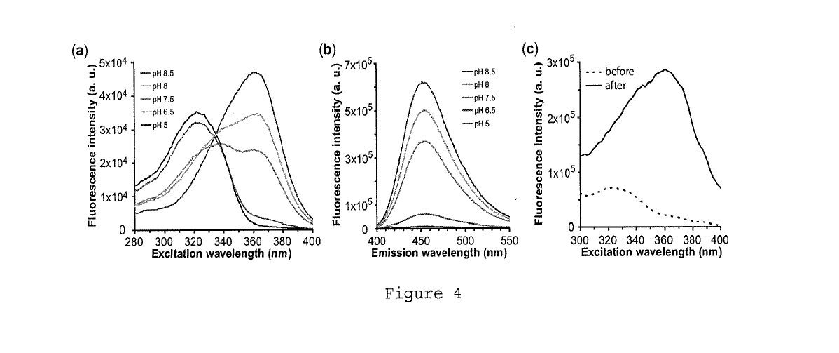

To understand the sensing mechanism, we measured the fluorescence spectra of 7HC at different pH in aqueous buffer (FIG. 4a). Consistent with 7-hydroxycoumarin (25), 7HC showed an excitation peak at 325 nm at low pH corresponding to the neutral phenol form, and at 365 nm at high pH corresponding to the anionic phenolate form. The excitation peak for 7HC-STAT3β shifted from 325 nm to 365 nm upon phosphorylation (FIG. 4c), consistent with the pH induced excitation shift of 7HC. In addition, when excited at a wavelength longer than the isosbestic point (335 nm), the emission intensity of 7HC increased with the pH (FIG. 4b) due to higher concentration of the anionic phenolate species at the ground state. Under similar excitation conditions, the fluorescence intensity of 7HC-STAT3β also increased after phosphorylation, suggesting a local pH increase. Both the shifted excitation peak and increased intensity of 7HC-STAT3β consistently suggest that the pH around 7HC increased upon phosphorylation. This pH increase results in deprotonation of phenolic 7HC in the 7HC-STAT3β to the phenolate form, which may occur due to an altered local hydrogen-bonding network induced by the incoming phosphate group. Moreover, crystal structures of the unphosphorylated and phosphorylated STAT3 protein show almost no conformational change after phosphorylation of Tyr705 (31), suggesting that a conformational change upon pTyr705 binding to the SH2 domain is not responsible for the observed 7HC fluorescence change.

Another unique fluorescence feature of 7HC-STAT3β is the appearance of an emission peak at 416 nm after phosphorylation, which provides a characteristic readout and has not been reported in other proteins containing 7HC (27). This emission peak corresponds to the excited state of the neutral phenol form of 7HC (32). When 7-hydroxycoumarin is excited in aqueous solution above pH 2, only a single emission peak at 456 nm corresponding to the excited phenolate species is observed regardless of which ground species is excited (25). We observed the same for 7HC in aqueous buffer (FIG. 4b). This is due to rapid deprotonation of the neutral phenol form of 7-hydroxycoumarin at the excited state, which occurs within the lifetime of the singlet excited state in aqueous solution (25). 7-hydroxycoumarin has also been excited in H₂O mixed with other solvents that are less efficient proton acceptors than H₂O (33). In such solvent mixture, when the mole fraction of H₂O decreases, the emission peak corresponding to the excited neutral phenol form of 7-hydroxycoumarin increases. Therefore, this emission peak is indicative of the inaccessibility of the fluorophore to H₂O. In the 7HC-STAT3β protein, a single emission peak corresponding to the phenolate form was observed before phosphorylation (FIGS. 3a and 3b), suggesting that 7HC has access to water and thus deprotonates very quickly at the excited state. However, the 416 nm emission peak corresponding to the neutral phenol form of 7HC emerged after phosphorylation (FIG. 3a). This indicates that deprotonation of the phenol form at the excited state is no longer rapid and that 7HC becomes shielded from water, possibly due to pTyr705 and its neighboring residues filling the SH2 pocket.

### EXAMPLE 5

### Reporting STAT3 Phosphorylation in HepG2 Cell Lysates

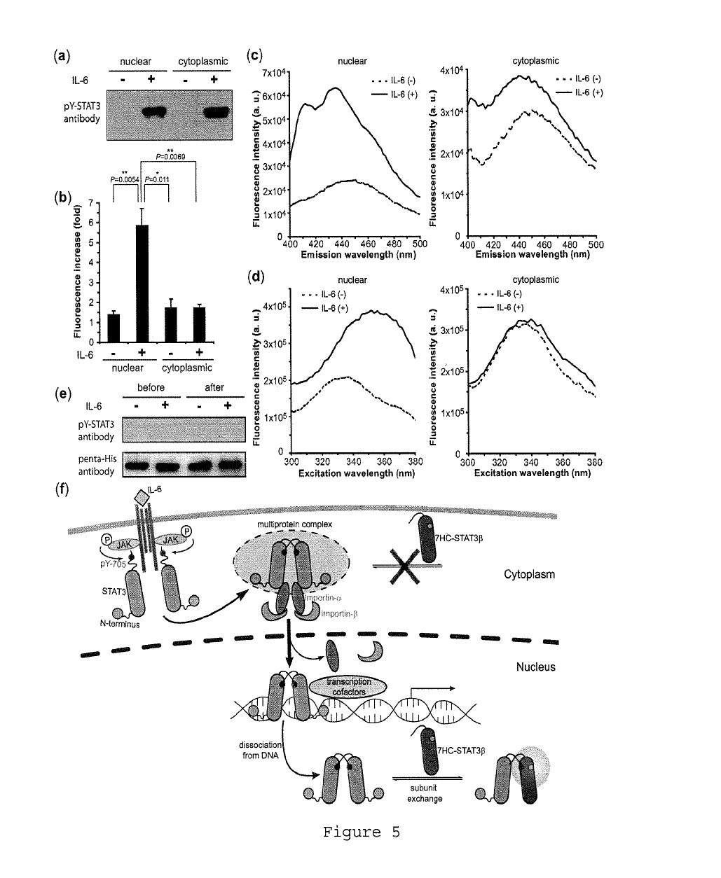

To test if 7HC-STAT3β can report the phosphorylation status of STAT3 proteins in mammalian cellular media, we incubated 7HC-STAT3β with cell lysates from human hepatoma HepG2 cells. A potent physiological activator of STAT3 is the cytokine interleukin-6 (IL-6), which signals through its cytokine receptor (34). HepG2 cells express both endogenous STAT3 and IL-6 receptor constitutively. Upon IL-6 binding, STAT3 is phosphorylated at Tyr705 by the receptor-associated and activated Janus kinase. Consistent with a previous report (34), we detected a high level of phosphorylated STAT3 in both the nuclear and cytoplasmic fraction of HepG2 cells treated with IL-6, but not in HepG2 cells in the absence of IL-6 activation (FIG. 5a). We then incubated the same amount of 7HC-STAT3β with independently prepared cell lysates and measured the fluorescence intensity. The fluorescence intensity increased 1.4 and 1.7 fold for the nuclear and cytoplasmic fractions, respectively, of cells that did not receive IL-6 (FIG. 5b). In contrast, for cells treated with IL-6, the fluorescence intensity of the nuclear fraction increased 5.9 fold, indicating that the 7HC-STAT3β can indeed optically report the phosphorylation status of endogenous STAT3. Unexpectedly, the fluorescence intensity increased only 1.7 fold for the cytoplasmic fraction of IL-6 treated cells, although it contained the same amount of phosphorylated STAT3. Thus, the extent of fluorescence increase was not significantly different among cell lysates of uninduced cells and the cytoplasmic fraction of induced cells, but significantly higher for the nuclear fraction of induced cells.

To understand the observed difference, we analyzed the emission spectra of the cell lysate samples after incubation with 7HC-STAT3β (FIG. 5c). Only the nuclear fraction of IL-6 induced cells showed the double emission peak characteristic of 7HC-STAT3β phosphorylated by Src as seen in FIG. 3a. The other 3 samples showed the same emission spectra as unphosphorylated 7HC-STAT3β. Consistently, only the excitation spectra for the nuclear fraction from IL-6 induced cells showed a peak shift to longer wavelength as seen in FIG. 4c for phosphorylated 7HC-STAT3β; the other 3 samples had excitation spectra identical to unphosphorylated 7HC-STAT3β (FIG. 5d). These results indicate that only in the nuclear fraction of IL-6 induced cells did binding of 7HC-STAT3β to a phosphotyrosine 705 occur, yielding fluorescence increase. Two possibilities can lead to such binding: 1) 7HC-STAT3β is phosphorylated by endogenous kinases in the cell lysate, after which it forms a homodimer or a heterodimer with endogenous phosphorylated STAT3; 2) unphosphorylated 7HC-STAT3β forms a heterodimer with phosphorylated endogenous STAT3. To distinguish this, an anti-phosphotyrosine STAT3 antibody was used to probe 7HC-STAT3β incubated in the cell lysate samples. Phosphorylation of 7HC-STAT3β was not detected in any cell lysate sample (FIG. 5e). This result is consistent with the fact that the activated Janus kinase is constitutively associated with the cytokine receptor and thus removed with the membrane during the preparation of cell lysates (34). In addition, it is known that STAT3α and STAT3β isoforms can form homodimers and heterodimers with each other (35). Therefore, we conclude that after being added to the nuclear fraction of IL-6 induced cells, 7HC-STAT3β is not phosphorylated but forms a heterodimer with endogenous phosphorylated STAT3 protein, resulting in the expected fluorescence intensity increase, characteristic double emission peak, and excitation peak shift.

It is intriguing to observe that the nuclear but not the cytoplasmic fraction of IL-6 induced HepG2 cells showed fluorescence increase upon incubation with 7HC-STAT3β, although both fractions contained phosphorylated endogenous STAT3. This difference is not due to the interference with or inactivation of 7HC-STAT3β reporter by cytoplasmic components, because when Src kinase was added to the IL-6 induced HepG2 cytoplasmic fraction incubated with 7HC-STAT3β, fluorescence increase with double peak emission was detected. Accumulating evidence from studies using gel-filtration chromatography (36-37) and fluorescence relaxation spectroscopy (38) suggests that phosphorylated STAT3 associates with a variety of proteins to form multiprotein complexes with molecular mass of 200-400 kDa and 1-2 MDa in the cytoplasm, instead of existing as a simple STAT3 dimer as in the classical model for JAK-STAT signaling. The associated proteins include chaperones and proteins involved in membrane trafficking and nuclear importing (39). Our finding is consistent with and corroborates this new view: association with other proteins may prevent subunit exchange of 7HC-STAT3β with phosphorylated STAT3 to form a heterodimer in the cytoplasm (FIG. 5f). In contrast, after being transported into the nucleus, the phosphorylated STAT3 dimer dissociates from the cytoplasmic proteins and importins α and β and is free to bind specific DNA targets to drive gene expression, after which it dissociates from DNA and is dephosphorylated by nuclear protein tyrosine phosphatases (40-41). The phosphorylated STAT3 is more accessible for subunit exchange with the reporter 7HC-STAT3β in the nucleus. To further identify those proteins that interact with STAT3 directly, in cellulo photocrosslinking using genetically encoded unnatural amino acids (42) may prove useful in this regard. Although our results indicate that it is difficult to exchange 7HC-STAT3β with the STAT3 subunit in the preformed cytoplasmic phosphorylated STAT3 complex in vitro, if 7HC-STAT3β is genetically coexpressed with endogenous STAT3 proteins inside cells, it should become phosphorylated and form dimers with endogenous phosphorylated STAT3 in situ to report phosphorylation status in live cells.

We developed a fluorescence reporter for the phosphorylation status of STAT3 by genetically incorporating the fluorescent unnatural amino acid 7HC into a selected site in STAT3. The reporter yielded fluorescence increase in response to phosphorylation by Src kinase and to endogenously phosphorylated STAT3 proteins. The reporter further revealed that there is a difference between the cytoplasmic and nuclear fractions of phosphorylated STAT3 in IL-6 activated HepG2 cells.

This method is genetically encodable and provides high sensitivity, thus combining the advantages of protein-based and peptide-based kinase reporters. Large fluorescence response was observed for nM concentrations of STAT3β, in contrast to μM concentrations needed for peptide-based kinase sensors. As Trp564 is conserved in all 7 mammalian STAT proteins (26), this method should be transferable to detect the phosphorylation of other STATs, which will be valuable to untangle the function of different STATs and various STAT isoforms selectively. A similar strategy can be applied to various SH2 domain-containing proteins, which participate in a variety of signal transduction pathways. A reporter based on the full-length substrate protein represents cellular characteristics of the target protein with high fidelity, and can also be used for reporting kinase as well as phosphatase activity with high specificity. In some embodiments, the methods described herein employ mammalian cells, and comprise the use of an orthogonal tRNA-synthetase pair that will enable the genetic incorporation of 7HC into proteins in the mammalian cells.

### EXAMPLE 6

### Methods

### Materials

DH10B E. coli cells (Invitrogen) were used for cloning and DNA preparation. Rosetta-2 (DE3) E. coli cells (Novagen) were used for protein production. Phusion™ high-fidelity DNA polymerase (New England Biolabs) was used for polymerase chain reaction (PCR). 7HC was synthesized as described (27). All other chemicals were purchased from Sigma-Aldrich.

### Expression System

All plasmids were assembled by standard cloning methods and confirmed by DNA sequencing. The plasmid pAIO-7HC encodes 1) the optimized tyrosyl amber suppressor tRNA_{CUA}^{Opt} (28) gene flanked by the lpp promoter and the rrnC terminator, 2) the 7HC-specific synthetase gene flanked by a modified E. coli glnS promoter and the glnS terminator, and 3) the mouse STAT3β gene flanked by the T5 promoter (followed by the lac operator) and the λ tₒ terminator. The gene cassette containing lpp promoter—JY17 tRNA—rrnC terminator was amplified from pYC-J17 (43) using primers 5′-AAC GGA TCC CGC CGC TTC TTT GAG-3′ and 5′-AAC GGA TCC AAA AAA AAT CCT TAG CTT TCG-3′. The PCR product was digested with BamH I, and ligated into pBK-JYRS (43) precut with BamH I to make pBK-J17-JYRS. A gene cassette containing the Hisx6 tag followed by the stop codon TAA and the λ tₒ terminator was made with primers 5′-AAG ATC TCA TCA CCA TCA CCA TCA CTA AGC TTA ATT AGC TGA GCT TGG ACT CC-3′ and 5′-GCT CGA GCA TGC TTG GAT TCT CAC CAA TAA AAA AC-3′. It was digested with Bgl II and Sph I, and cloned into pBK-J17-JYRS precut with the same enzymes to make pBK-J17-JYRS-Hisx6. To change tRNA J17 into the optimized tRNA_{CUA}^{Opt}, QuikChange (Strategene) was performed with primers QCtRNA.AGGf1 (5′-CTC TAA ATC CGC ATG GCA GGG GTT CAA ATC CGG CCC G) and QCtRNA.AGGr1 (5′-CGG GCC GGA TTT GAA CCC CTG CCA TGC GGA TTT AGA G), and then with primers QCtRNA.CCTf2 (5′-GGC AGG GGT TCA AAT CCC CTC CGC CGG ACC AC) and QCtRNA.CCTr2 (5′-GTG GTC CGG CGG AGG GGA TTT GAA CCC CTG CC-3′), resulting in plasmid pBK-tRNA^{Opt}-JYRS-Hisx6. The 7HC specific synthetase gene was digested from pEB-CouRS (a gift from Drs. Eli Chapman and Peter G. Schultz) using Nde I and Stu I, and ligated into plasmid pBK-tRNA^{Opt}-JYRS-Hisx6 precut with the same restriction enzymes to make pBK-tRNA^{Opt}-CouRS-Hisx6. The N-terminal 126 residues of the STAT3β were truncated, which does not affect DNA binding and phosphorylation (26). The STAT3β gene cassette was generated from a mouse STAT3 α cDNA using two rounds of PCR to introduce the N-terminal truncation, C-terminal truncation, and C-terminal addition of the 7 amino acids unique to STAT3β and a thrombin cleavage site. The first PCR used primers F1 (5′-ATT CCA TAT GGG CCA GGC CAA CCA CCC AAC AG-3′) and R1 (5′-CCA CGT GGC ACC AAT TTC CAA ACT GCA TCA ATG AAT GGT GTC ACA CAG ATG AAC-3′). The second PCR used primers F1 and R2 (5′-CCT AAG CTT TGA TCC ACG TGG CAC CAA TTT CC-3′). The PCR product was ligated into p-GEM (Promega) using the manufacturer's instructions. Using this p-GEM product as the template, two more rounds of PCR were performed to append the T5 promoter upstream of the STAT3β gene with primers F2 (5′-GTA TAA TAG ATT CAT AAA TTT GAT TAA AGA GGA GAA ATT AAC TAT GGG CCA GGC CAA CCA C-3′) and R3 (5′-CCA CGT AGA TCT TGA TCC ACG TGG CAC CAA TTT CC-3′), followed by primers F3 (5′-CAG CTC GGG CCC TTG CTT TCA GGA AAA TTT TTC AGT ATA ATA GAT TCA TA-3′) and R3. The PCR product was digested with Apa I and Bgl II, and ligated into plasmid pBK-tRNA^{Opt}-CouRS-Hisx6 digested with the same enzymes to afford the final plasmid pAIO-7HC. All STAT3β mutants were made by site-directed mutagenesis using the QuikChange kit (Strategene). For mutant Y705F, the primers were 5′-GTA GTG CTG CCC CGT TCC TGA AGA CCA AG-3′ and 5′-CTT GGT CTT CAG GAA CGG GGC AGC ACT AC-3′. For mutant R609Q, the primers were 5′-GCA CCT TCC TAC TGC AGT TCA GCG AGA GCA GC-3′ and 5′-GCT GCT CTC GCT GAA CTG CAG TAG GAA GGT GC-3′.

### Protein Expression and Purification

The plasmid pAIO-7HC was transformed into Rosetta-2 cells harboring the pRARE-2 plasmid. Small starter cultures (20 mL) were grown overnight to saturation in 2×YT. These were used to seed larger 250 mL cultures in LB with a starting O.D.₆₀₀ of 0.05. Cultures were grown at 37° C. until O.D. reached 0.2-0.3, and 1 mM 7-HC was added. Once the O.D. reached 0.5, cultures were moved to an 18° C. shaker. After 30 mins cultures were induced with 0.5 mM IPTG and grown an additional 20 hrs. Cultures were spun at 5000 rpm (4225 RCF) 10 mins to pellet cells. Pellets were resuspended in 5 mL Buffer A plus EDTA-free protease inhibitor (Roche), 0.5 mg/mL lysozyme, and DNAse. Buffer A contained the following: 5% glycerol, 0.1% Tween, 5 mM βME, 300 mM NaCl, 50 mM NaH₂PO₄/Na₂HPO₄ buffer (pH 7.5). After rocking at 4° C. for 1 hour, the cells were sonicated with a microtip. Lysates were spun at 35,000 RCF for 20 mins, and incubated with 250 μL Ni-NTA resin (Qiagen) pre-equilibrated in Buffer A at 4° C. for 1 hour. Ni-NTA resin was washed 3 times with 10 mL Buffer A plus 20 mM imidazole. Protein was eluted in fractions of 250 μL Buffer A plus 250 mM imidazole. Fractions 1-3 were combined and dialyzed overnight in 1 L of 20 mM Tris buffer containing 100 mM NaCl (pH 7.0). The purified protein was divided into aliquots and stored at −80° C.

### Phosphorylation Reactions

Purified protein was incubated with Src kinase (Sigma) in kinase buffer plus 1 mM ATP for 30 min at 30° C. The kinase buffer contained the following: 5 mM HEPES (pH 7.5), 5 mM MgCl₂, 5 mM MnCl₂, 1.25 mM DTT, 3 μM Na₂VO₄. The amount of Src kinase (0.07 nM to 0.7 nM) used was >100 fold less than the amount of STAT3 protein (8.6 nM to 86 nM). Reactions were stopped with 20 mM EDTA.

### Western Blot

Proteins from phosphorylation reactions were loaded onto an 8% polyacrylamide gel, separated by electrophoresis, and transferred to a PVDF membrane. A penta-His antibody with HRP conjugate (Qiagen) was used for detecting the Hisx6 tagged STAT3β proteins, a p-Stat3(B-7) antibody (Santa Cruz, catalog No. sc-8059) for detecting Tyr705-phosphorylated STAT3 proteins, and a Stat3 (K-15) antibody (Santa Cruz, catalog No. sc-483) for detecting all STAT3 proteins.

### Mobility Shift Assay

A Single strand of hSIE (m67) probe was (γ-³²P)ATP (MP Biomedicals, LLC) end-labeled with T4 polynucleotide kinase (New England Biolabs) per the manufacturer's instructions and annealed with the complementary strand to form DNA duplex (final concentration ˜0.15 μM). The sequence of the hSIE probe is 5′-AGC TTC ATT TCC CGT AAA TCC CTA AAG CT-3′. The binding reaction in the final volume of 25 μL contained the following: 10 mM HEPES (pH 7.5), 50 mM KCl, 1 mM EDTA, 10% glycerol, 5 mM DTT, 0.5 mM PMSF, 1 mM Na₂VO₄, 1 μg poly(dI-dC), 7.5 μg BSA, 17.75 μL kinase reaction solution containing 120 ng STAT3 protein, and 0.075 pmol (γ-³²P) labeled hSIE probe. The binding reaction was carried out at RT for 35 mins, and 20 μL was immediately loaded onto 4% native polyacrylamide gel at 4° C. The gel was run in 0.5×TBE, dried, and exposed to a phosphor screen.

### Fluorometry

Kinase reactions were run in 150 μL total volume at 30° C. for 30 mins with components described in the section of phosphorylation reactions. For results in FIGS. 3a, 3b and 4c, 20 nM of 7HC-STAT3β was used and the experiments were performed independently 6 times. Calf intestinal alkaline phosphatase (CIP, 1 μL) (New England Biolabs) was added to some of the phosphorylated reactions to test the effect of dephosphorylation. All samples were then immediately recorded on Fluorolog-3 (Horiba Jobin Yvon) in a 100 μL quartz microcuvette. Samples were excited at 345 nm, and emission from 400-600 nm was recorded. The slit width was 4 nm for both excitation and emission. The negative control was prepared from Rosetta-2 (DE3) cells transformed with pAIO-7HC and pRARE2 but without 7-HC added into the growth media.

### HepG2 Cell Lysate Experiments

HepG2 cells were grown in DMEM containing 10% FBS, 1% penicillin-streptomycin and maintained at 37° C. and 5% CO₂. Cell activation was carried out as described previously using 0.5 nM recombinant human IL-6 (R&D Systems) (44). Cytoplasmic and nuclear fractions were prepared using the NE-PER kit (Thermo Scientific) according to the manufacturer's instructions. Western blot was used to confirm endogenous STAT3 activation. Cytoplasmic (7 μL) or nuclear fractions (7 μL) were loaded onto a 10% polyacrylamide gel, and blots were probed with the p-Stat3(B-7) antibody (Santa Cruz), which is specific for the Tyr705-phosphorylated STAT3. For fluorescence measurement, 7HC-STAT3β (135 ng) was incubated with ˜10 μL of cell lysate from three independent preparations of HepG2 cells in a total reaction volume of 130 μL in TBS (20 mM Tris, pH=7.5, 150 mM NaCl) at 30° C. The ˜10 μL of cell lysate contained 10-20 μg of total protein for the nuclear fractions or 150-160 μg of total protein for the cytoplasmic fractions. The volume of the cell lysate added was adjusted to contain the same amount of phosphorylated STAT3 for both the nuclear and cytoplasmic fractions based on the densitometry of Western blots. Readings were taken on the fluorometer (μₑₓ=350 nm, μₑₘ=400−500 nm, slit widths: Ex=4 nm, Em=4 nm) in a quartz microcuvette maintained at 30° C. at various time points up to 4 hours until the signal plateaued. To determine if endogenous kinase within the cell lysates phosphorylates the 7HC-STAT3β probe, 20 μL of the reactions used on the fluorometer was directly loaded on 10% polyacrylamide gels for Western blot analysis. Blots were probed with the penta-His antibody (Qiagen) to confirm the presence of 7HC-STAT3β and the p-Stat3(B-7) antibody for detecting phosphorylation.

### REFERENCES

1. Blume-Jensen, P., and Hunter, T. (2001) Oncogenic kinase signalling, Nature 411, 355-365.
2. Ortutay, C., Valiaho, J., Stenberg, K., and Vihinen, M. (2005) KinMutBase: a registry of disease-causing mutations in protein kinase domains, Hum. Mutat. 25, 435-442.
3. Knight, Z. A., Lin, H., and Shokat, K. M. (2010) Targeting the cancer kinome through polypharmacology, Nat. Rev. Cancer 10, 130-137.
4. Shults, M. D., and Imperiali, B. (2003) Versatile fluorescence probes of protein kinase activity, J. Am. Chem. Soc. 125, 14248-14249.
5. Shults, M. D., Janes, K. A., Lauffenburger, D. A., and Imperiali, B. (2005) A multiplexed homogeneous fluorescence-based assay for protein kinase activity in cell lysates, Nat. Methods 2, 277-283.
6. Yeh, R. H., Yan, X., Cammer, M., Bresnick, A. R., and Lawrence, D. S. (2002) Real time visualization of protein kinase activity in living cells, J. Biol. Chem. 277, 11527-11532.
7. Sharma, V., Agnes, R. S., and Lawrence, D. S. (2007) Deep quench: an expanded dynamic range for protein kinase sensors, J. Am. Chem. Soc. 129, 2742-2743.
8. Rothman, D. M., Shults, M. D., and Imperiali, B. (2005) Chemical approaches for investigating phosphorylation in signal transduction networks, Trends Cell Biol. 15, 502-510.
9. Sharma, V., Wang, Q., and Lawrence, D. S. (2008) Peptide-based fluorescent sensors of protein kinase activity: design and applications, Biochim. Biophys. Acta 1784, 94-99.
10. Zhang, J., Ma, Y., Taylor, S. S., and Tsien, R. Y. (2001) Genetically encoded reporters of protein kinase A activity reveal impact of substrate tethering, Proc. Natl. Acad. Sci. U.S.A. 98, 14997-15002.
11. Violin, J. D., Zhang, J., Tsien, R. Y., and Newton, A. C. (2003) A genetically encoded fluorescent reporter reveals oscillatory phosphorylation by protein kinase C, J. Cell Biol. 161, 899-909.
12. Ting, A. Y., Kain, K. H., Klemke, R. L., and Tsien, R. Y. (2001) Genetically encoded fluorescent reporters of protein tyrosine kinase activities in living cells, Proc. Natl. Acad. Sci. U.S.A. 98, 15003-15008.
13. Wang, Y., Botvinick, E. L., Zhao, Y., Berns, M. W., Usami, S., Tsien, R. Y., and Chien, S. (2005) Visualizing the mechanical activation of Src, Nature 434, 1040-1045.
14. Humphries, K. M., Pennypacker, J. K., and Taylor, S. S. (2007) Redox regulation of cAMP-dependent protein kinase signaling: kinase versus phosphatase inactivation, J. Biol. Chem. 282, 22072-22079.
15. Ouyang, M., Huang, H., Shaner, N. C., Remacle, A. G., Shiryaev, S. A., Strongin, A. Y., Tsien, R. Y., and Wang, Y. (2010) Simultaneous visualization of protumorigenic Src and MT1-MMP activities with fluorescence resonance energy transfer, Cancer Res. 70, 2204-2212.
16. Lippincott-Schwartz, J., Snapp, E., and Kenworthy, A. (2001) Studying protein dynamics in living cells, Nat. Rev. Mol. Cell Biol. 2, 444-456.
17. Zhang, J., Campbell, R. E., Ting, A. Y., and Tsien, R. Y. (2002) Creating new fluorescent probes for cell biology, Nat. Rev. Mol. Cell Biol. 3, 906-918.
18. Miyawaki, A. (2003) Visualization of the spatial and temporal dynamics of intracellular signaling, Dev. Cell 4, 295-305.
19. Zhang, J., and Allen, M. D. (2007) FRET-based biosensors for protein kinases: illuminating the kinome, Mol. Biosyst. 3, 759-765.
20. Manning, G., Whyte, D. B., Martinez, R., Hunter, T., and Sudarsanam, S. (2002) The protein kinase complement of the human genome, Science 298, 1912-1934.
21. Remenyi, A., Good, M. C., and Lim, W. A. (2006) Docking interactions in protein kinase and phosphatase networks, Curr. Opin. Struct. Biol. 16, 676-685.
22. Lukovic, E., Gonzalez-Vera, J. A., and Imperiali, B. (2008) Recognition-domain focused chemosensors: versatile and efficient reporters of protein kinase activity, J. Am. Chem. Soc. 130, 12821-12827.
23. Lawrence, D. S., and Wang, Q. (2007) Seeing is believing: peptide-based fluorescent sensors of protein tyrosine kinase activity, Chem Bio Chem 8, 373-378.
24. Yu, H., Pardoll, D., and Jove, R. (2009) STATs in cancer inflammation and immunity: a leading role for STAT3, Nat. Rev. Cancer 9, 798-809.
25. Fink, D. W., and Koehler, W. R. (1970) pH effects on fluorescence of umbelliferone, Anal. Chem. 42, 990-993.
26. Becker, S., Groner, B., and Muller, C. W. (1998) Three-dimensional structure of the Stat3beta homodimer bound to DNA, Nature 394, 145-151.
27. Wang, J., Xie, J., and Schultz, P. G. (2006) A genetically encoded fluorescent amino acid, J. Am. Chem. Soc. 128, 8738-8739.
28. Young, T. S., Ahmad, I., Yin, J. A., and Schultz, P. G. (2009) An enhanced system for unnatural amino acid mutagenesis in E. coli, J. Mol. Biol. 395, 361-374.
29. Kretzschmar, A. K., Dinger, M. C., Henze, C., Brocke-Heidrich, K., and Horn, F. (2004) Analysis of Stat3 (signal transducer and activator of transcription 3) dimerization by fluorescence resonance energy transfer in living cells, Biochem. J. 377, 289-297.
30. Mayer, B. J., Jackson, P. K., Van Etten, R. A., and Baltimore, D. (1992) Point mutations in the abl SH2 domain coordinately impair phosphotyrosine binding in vitro and transforming activity in vivo, Mol. Cell. Biol. 12, 609-618.
31. Ren, Z., Mao, X., Mertens, C., Krishnaraj, R., Qin, J., Mandal, P. K., Romanowski, M. J., McMurray, J. S., and Chen, X. (2008) Crystal structure of unphosphorylated STAT3 core fragment, Biochem. Biophys. Res. Commun. 374, 1-5.
32. Moriya, T. (1983) Excited-state reactions of coumarins in aqueous solutions. I. The phototautomerization of 7-hydroxycoumarin and its derivative, Bull. Chem. Soc. Jpn. 56, 6-14.
33. Zinsli, P. E. (1974) Investigation of rate parameters in chemical reactions of excited hydroxycoumarins in different solvents, J. Photochem. 3, 55-69.
34. Lutticken, C., Wegenka, U. M., Yuan, J., Buschmann, J., Schindler, C., Ziemiecki, A., Harpur, A. G., Wilks, A. F., Yasukawa, K., Taga, T., and et al. (1994) Association of transcription factor APRF and protein kinase Jak1 with the interleukin-6 signal transducer gp130, Science 263, 89-92.
35. Park, O. K., Schaefer, L. K., Wang, W., and Schaefer, T. S. (2000) Dimer stability as a determinant of differential DNA binding activity of Stat3 isoforms, J. Biol. Chem. 275, 32244-32249.
36. Ndubuisi, M. I., Guo, G. G., Fried, V. A., Etlinger, J. D., and Sehgal, P. B. (1999) Cellular physiology of STAT3: Where's the cytoplasmic monomer?, J. Biol. Chem. 274, 25499-25509.
37. Shah, M., Patel, K., Mukhopadhyay, S., Xu, F., Guo, G., and Sehgal, P. B. (2006) Membrane-associated STAT3 and PY-STAT3 in the cytoplasm, J. Biol. Chem. 281, 7302-7308.
38. Watanabe, K., Saito, K., Kinjo, M., Matsuda, T., Tamura, M., Kon, S., Miyazaki, T., and Uede, T. (2004) Molecular dynamics of STAT3 on IL-6 signaling pathway in living cells, Biochem. Biophys. Res. Commun. 324, 1264-1273.
39. Kawashima, T., Bao, Y. C., Minoshima, Y., Nomura, Y., Hatori, T., Hori, T., Fukagawa, T., Fukada, T., Takahashi, N., Nosaka, T., Inoue, M., Sato, T., Kukimoto-Niino, M., Shirouzu, M., Yokoyama, S., and Kitamura, T. (2009) A Rac GTPase-activating protein, MgcRacGAP, is a nuclear localizing signal-containing nuclear chaperone in the activation of STAT transcription factors, Mol. Cell. Biol. 29, 1796-1813.
40. Reich, N. C., and Liu, L. (2006) Tracking STAT nuclear traffic, Nat. Rev. Immunol. 6, 602-612.
41. Mertens, C., and Darnell, J. E., Jr. (2007) SnapShot: JAK-STAT signaling, Cell 131, 612.
42. Takimoto, J. K., Adams, K. L., Xiang, Z., and Wang, L. (2009) Improving orthogonal tRNA-synthetase recognition for efficient unnatural amino acid incorporation and application in mammalian cells, Mol. Biosyst. 5, 931-934.
43. Wang, L., Brock, A., Herberich, B., and Schultz, P. G. (2001) Expanding the genetic code of Escherichia coli, Science 292, 498-500.
44. Draber, P., Draberova, L., Heneberg, P., Smid, F., and Farghali, H. (2007) Preformed STAT3 transducer complexes in human HepG2 cells and rat hepatocytes, Cell. Signal. 19, 2400-2412.

Having thus described embodiments of the present invention, it is to be understood that the invention defined by the above paragraphs is not to be limited to particular details set forth in the above description as many apparent variations thereof are possible without departing from the spirit or scope of the present invention.

Each patent, patent application, and publication cited or described in the present application is hereby incorporated by reference in its entirety as if each individual patent, patent application, or publication was specifically and individually indicated to be incorporated by reference.

1. A method for the detection of a modification to a target protein, comprising:
(a) contacting a target protein comprising an unnatural amino acid with a modifying enzyme; and
(b) assaying for a detectable signal from the unnatural amino acid in (a) after the target protein of (a) has been contacted with the modifying enzyme,
wherein if the modifying enzyme modifies the target protein, the unnatural amino acid generates a detectable signal, thereby indicating that the target protein has been modified.

2. The method of claim 1, wherein the detectable signal is fluorescence.

3. The method of claim 1, wherein the unnatural amino acid is 7HC.

4. The method of claim 1, wherein the target protein is STAT3.

5. The method of claim 1, wherein the target protein comprises an SH2 domain.

6. The method of claim 1, wherein the modifying enzyme is a kinase.

7. The method of claim 1, wherein the modification to the target protein is phosphorylation.

8. The method of claim 1, wherein the target protein comprising the unnatural amino acid is expressed in a host cell selected from the group consisting of bacterial, insect, and mammalian cells.

9. The method of claim 8, wherein the host cell is a bacterial cell.

10. The method of claim 9, wherein the bacterial cell is an E. coli cell.

11. The method of claim 8, wherein the host cell is a mammalian cell.

12. The method of claim 11, wherein the mammalian cell is a HepG2 cell.

13. The method of claim 1, wherein the unnatural amino acid generates a detectable signal as a result of a conformational change.

14. The method of claim 1, wherein the unnatural amino acid generates a detectable signal as a result of a pH change.

15. The method of claim 8, further comprising contacting the host cell expressing the target protein with a physiological activator of the target protein.

16. The method of claim 15, wherein the target protein is STAT3 and the physiological activator is IL-6.
